(19) United States
(12) Patent Application Publication
(10) Pub. No.: US 2010/0217320 A1
(43) Pub. Date: Aug. 26, 2010

(54) VERTEBRAL IMPLANT MADE FROM BONE MATERIAL FOR RELIEF OF A NARROWED VERTEBRAL CHANNEL

(75) Inventor: Klaus Landis, Heroldsberg (DE)

Correspondence Address: GIFFORD, KRASS, SPRINKLE,ANDERSON & CITKOWSKI, P.C, PO BOX 7021, TROY, MI, 48007-7021, US

(73) Assignee: TUTOGEN MEDICAL GMBH

(21) Appl. No.: 11/917,954

(22) PCT Filed: May 3, 2006
(86) PCT No.: PCT/EP06/04133

§ 371 Date: Feb. 29, 2008

(30) Foreign Application Priority Data
Jun. 22, 2005 (DE) 10 2005 028 887.1

(51) Int. Cl. A61B 17/70

(52) U.S. Cl. 606/249

(57) ABSTRACT

The invention relates to an implant for relief of a narrowed vertebral channel in a spinal column, with a spacer for application between the spinous processes of two adjacent vertebral bodies and two fixing elements arranged to the left and right of the spinous processes in the implanted state. The implant is made from bony material and is formed from at least two pieces, one piece having a sleeve section formed in one piece with one fixing element the other piece having a plug section formed in one piece with the other fixing element, both sections being able to be connected to each other in the form of a plug connection, the sleeve section and/or the plug section being part of the spacer or a part thereof.

The invention relates to an implant for the relief of a narrowed spinal canal of a spinal column having a spacer which is insertable between the spinous processes of two adjacent vertebral bodies and having two fixing elements which are arranged to the left and to the right of the spinous processes in the implanted state.

In the event of spinal canal stenosis, a narrowing of the spinal canal occurs so that the function of the nerves extending in the spinal canal is impaired. The narrowing can, for example, be caused by degenerate vertebral disks which penetrate into the spinal canal and press onto the nerves extending there. Back pain can occur as a consequence of such a constriction which can also radiate into the legs and a feeling of numbness can occur in the buttocks and/or in the legs.

It is known for the treatment of spinal canal stenosis to insert an implant between the spinous processes of the affected spinal section. A spacer of the implant presses the two spinous processes of the affected vertebral bodies apart so that the spinal canal is permanently widened and relieved. To secure the spacer against slippage, the implant has two fixing elements which are arranged to the left and to the right of the spinous processes in the implanted state.

Such known implants consist of titanium and are therefore a foreign body in the human body, on the one hand, and comparatively expensive, on the other hand.

It is the underlying object of the invention to provide an implant of the initially named kind which is particularly compatible, can be manufactured cost-effectively and can simultaneously be handled, in particular assembled, easily.

This object is satisfied by the features of claim 1 and in particular in that the implant consists of bone material and is made in at least two parts, with the one part including a socket-like section made in one piece with the one fixing element and the other part including a plug-like section made in one piece with the other fixing element, the two sections being connectable to one another in the manner of a plug connection and the socket-like section and/or the plug-like section being the spacer or a part thereof.

A particular advantage of the implant in accordance with the invention is provided by the material used which, on the basis of its biological origin, does not represent a foreign body. An implant can furthermore be manufactured more cost-effectively from bone material than is the case with an implant made of titanium.

The implant includes at least two parts. The one part of the implant includes a socket-like section and the other part includes a suitably made plug-like section so that the two sections can be plugged into one another or together. The two parts can hereby be connected to one another in a particularly simple manner without further individual parts such as screws or the like being necessary which frequently result in premature loosening on a connection to bone parts.

The implant in accordance with the invention is configured such that the socket-like section and/or the plug-like connection represents the spacer, which is in particular elongated and can be inserted between the spinous processes of the two affected vertebral bodies, or a part thereof. The plug-like section can, for example, be arranged substantially completely within the socket-like section with an assembled implant so that the socket-like section acts as a spacer. Alternatively, the socket-like section can be made particularly short with respect to the plug-like section so that the plug-like section projects sufficiently beyond the socket-like section, and thus forms the spacer, with an assembled implant.

Advantageous embodiments of the invention are also recited in the dependent claims, in the description and in the drawing.

The socket-like section can be made in the manner of a sleeve or of a hollow cylinder and/or the plug-like section can be made in the manner of a spigot or of a pin. An inner surface of the socket-like section and an outer surface of the plug-like section can in particular each be made conically to form a pressed fit with an assembled implant. A plug connection is preferably to be understood as a connection in which a socket-like section and a plug-like section can in particular be plugged into one another at least partly so that at least parts of the socket-like section mutually surround at least parts of the plug-like sections for a holding of the two sections.

In accordance with a preferred embodiment of the invention, at least one of the fixing elements is made as an abutment for the socket-like section and/or the plug-like section on the plugging together of the sections, i.e. the fixing element connected to the plug-like section can be made as an abutment for the socket-like section and/or the fixing element connected to the socket-like section can be made as an abutment for the plug-like section. The two sections can hereby be correctly assembled in a simple manner. The length of the spacer then corresponds to the length of the socket-like section and/or of the plug-like section.

To ensure a particularly firm and secure connection between the two sections, the assembled sections can be adhesively bonded to one another in addition to the plug connection. A biocompatible acrylic adhesive can be used for this, for example.

At least one of the two parts is preferably made in T shape and/or the assembled implant is made in H shape, viewed in cross-section. The H shape can be achieved by putting two T-shaped parts together. An H shape has proved to be particularly suitable with respect to the prevention of slippage of the implanted implant.

On the implanting, the ligament formed between the spinous processes must be pierced to enable the insertion of the spacer. It is therefore particularly advantageous if means, in particular a metal tip, can be placed onto at least one of the two parts, in particular a section, for the piercing of the ligament formed between the spinous processes. The means can in particular have a plug projection which can be plugged together with the socket-like section. It can hereby be achieved that, simultaneously with the piercing of the belt, the part carrying the piercing means, in particular its section, can be guided through the ligament formed between the spinous processes.

The invention furthermore relates to an implant system having an implant as is explained above and having means for the piercing of the ligament formed between the spinous processes. The piercing means can be placed onto at least one of the two parts. The piercing means can, however, also be a tool which cannot be connected to the parts of the implant and which first pierces the ligament formed between the spinous processes on the implanting before the implant in accordance with the invention is guided through the ligament in a next step.

The invention will be explained in the following by way of example with reference to the drawing. There are shown in it:

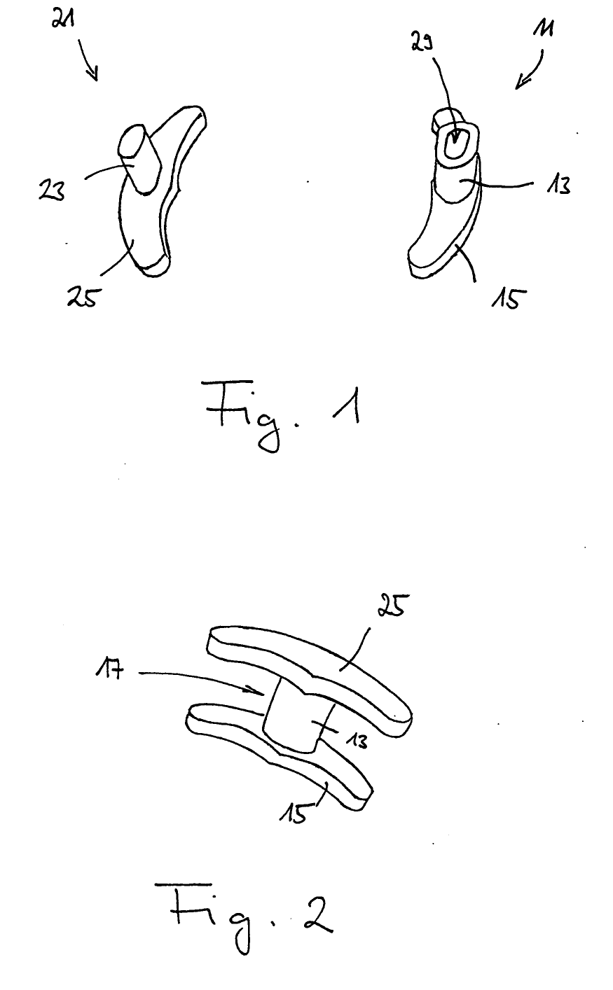

FIG. 1 a perspective view of the two parts of the implant in accordance with the invention;

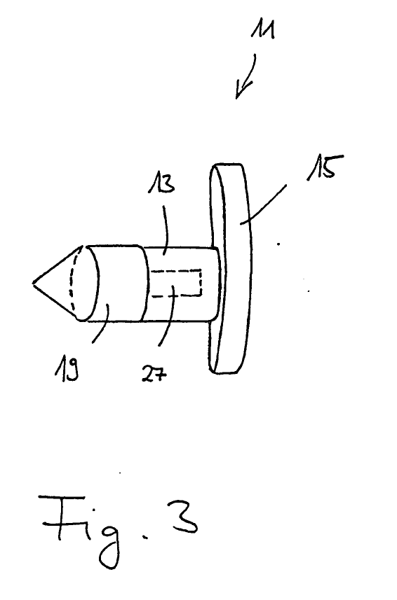

FIG. 2 a perspective view of the implant in accordance with the invention assembled from the two parts of FIG. 1; and

FIG. 3 a perspective view of a part of FIG. 1 with a metal tip placed on.

FIG. 1 shows an implant in accordance with the invention for the relief of a narrowed spinal canal of a spinal column in a non-assembled representation. The implant includes two parts 11, 21 which each consist of bone material. The one part 11 has a socket-like section 13 which is made in one piece with a wing-like fixing element 15. The socket-like section is made as a hollow cylinder 13 with an oval cross-section. The other part 21 has a plug-like section 23 which is made in one piece with a wing-like fixing element 25 which is like the wing-like fixing element 15. The plug-like section is made as a spigot 23 with an oval cross-section. Each of the two parts 11, 21 has an essentially T-like shape in a projection into a plane.

The two sections 13, 23 of the two parts 11, 21 can be connected to one another in the manner of a plug connection, with the assembled implant having an H shape (FIG. 2). The plug connection is in particular made possible in that the size and external shape of the spigot 23, which is arranged within the hollow cylinder 13 in the assembled state of the implant and can consequently not be shown in FIG. 2, are matched to the size and the external shape of the opening 29 (FIG. 1) formed through the hollow cylinder into which the spigot 23 engages. In addition to the plug connection, the two plugged together sections 13, 23 are adhesively bonded to one another by means of a biocompatible acrylic adhesive.

The longitudinal extent of the hollow cylinder 13 substantially corresponds to the longitudinal extent of the spigot 23 so that, on the assembly of the two parts 11, 21, the fixing element 25 serves as an abutment for the hollow cylinder 13 and the fixing element 15 serves as an abutment for the spigot 23.

The hollow cylinder 13, which completely surrounds the spigot 23 in the radial direction in the assembled state of the implant in accordance with the invention, acts as a spacer 17 which is insertable between the spinous processes of two adjacent vertebral bodies with a narrowed spinal canal. The two fixing elements 15, 25 are then arranged to the left and to the right of the spinous processes in the implanted state. A particularly good support and fixing of the implant in the body in accordance with the invention can be ensured by the wing-like shape of the fixing elements 15, 25 so that no additional fixing of the implant to the bone or ligaments with sutures, wires or the like is necessary.

Since a ligament is formed between the two spinous processes between which the spacer 17 should inserted, the ligament must be pierced regionally to enable a guiding through of the spacer 17. A metal tip 19 is provided for this purpose in accordance with FIG. 3 which has at its side disposed opposite the tip a plug projection 27 with which the metal tip 19 can be inserted into the opening 29 formed by the hollow cylinder 13 (FIG. 1).

The insertion of the implant in accordance with the invention between the spinous processes of two adjacent vertebral bodies will be described in the following with reference to the drawing.

The metal tip 19 is first placed onto the hollow cylinder 13 of the part 11 of the implant in accordance with the invention. Then the ligament formed between the spinous processes is pierced by the metal tip 19 at the front and the hollow cylinder 13 is thus pushed through the ligament and placed between the spinous processes of the vertebral bodies. Subsequently, the metal tip 19 is removed and the part 21 having the spigot 23 is placed on from the side of the ligament disposed opposite the part 11 by introducing the spigot 23 into the hollow cylinder 13.

To increase the holding force between the two parts 11, 21 or sections 13, 23, the outer cylinder jacket of the spigot 23, the inner cylinder jacket of the hollow cylinder 13, the end face of the spigot 23 facing the fixing element 25 and/or the end face of the hollow cylinder 13 remote from the fixing element 15 are coated with a biocompatible acrylic adhesive before the plugging together.

### REFERENCE NUMERAL LIST

11 part
13 hollow cylinder
15 fixing element
17 spacer
19 metal tip
21 part
23 spigot
25 fixing element
27 plug projection
29 opening

1. An implant for the relief of a narrowed spinal canal of a spinal column having a spacer (17) which is insertable between the spinous processes of two adjacent vertebral bodies and having two fixing elements (15, 25) which are arranged to the left and to the right of the spinous processes in the implanted state, said implant being made in at least two parts, wherein
the one part (11) including a socket-like section (13) which is made in one piece with the one fixing element (15);
the other part (21) including a plug-like section (23) which is made in one piece with the other fixing element (25);
the two sections (13, 23) being connectable to one another in the manner of a plug connection; and
the socket-like section (13) and/or the plug-like section (23) being the spacer (17) or a part thereof;
characterized in that
the implant is made of bone material, with
the socket-like section (13) being made as a hollow cylinder with an oval cross-section;
the plug-like section (23) being made as a spigot with an oval cross-section; and
the plugged-together sections (13, 23) being adhesively bonded with one another.

2. (canceled)

3. An implant in accordance with claim 1, characterized in that at least one of the fixing elements (15, 25) is made as an abutment for the socket-like section and/or plug-like section (13, 23) on the assembly of the sections (13, 23).

4. An implant in accordance with claim 1, characterized in that the plugged together sections (13, 23) are adhesively bonded to one another by means of a biocompatible acrylic adhesive.

5. An implant in accordance with claim 1, characterized in that at least one of the two parts (11, 21) is made in T-shape and/or the assembled implant is H-shaped viewed in cross-section.

6. An implant in accordance with claim 1, characterized in that means (19), in particular a metal tip, can be placed onto at least one of the two parts (11, 21) for the piercing of the ligament formed between the spinous processes.

7. An implant system comprising an implant for the relief of a narrowed spinal canal of a spinal column having a spacer (17) which is insertable between the spinous processes of two adjacent vertebral bodies and having two fixing elements (15, 25) which are arranged to the left and to the right of the spinous processes in the implanted state, said implant being made in at least two parts, wherein
the one part (11) including a socket-like section (13) which is made in one piece with the one fixing element (15);
the other part (21) including a plug-like section (23) which is made in one piece with the other fixing element (25);
the two sections (13, 23) being connectable to one another in the manner of a plug connection; and
the socket-like section (13) and/or the plug-like section (23) being the spacer (17) or a part thereof;
characterized in that
the implant is made of bone material, with
the socket-like section (13) being made as a hollow cylinder with an oval cross-section;
the plug-like section (23) being made as a spigot with an oval cross-section; and
the plugged-together sections (13, 23) being adhesively bonded with one another;
and comprising means (19) for the piercing of the ligament formed between the spinous processes.
